(19) United States
(12) Patent Application Publication
(10) Pub. No.: US 2010/0069298 A1
(43) Pub. Date: Mar. 18, 2010

(54) Methods for Detecting Overexpression of SPARC and Therapeutic and Diagnostic Methods Relating to Same

(76) Inventors: Robert Penny, Lebanon, IN (US); Julie LaRocca, Phoenix, AZ (US)

Correspondence Address: Caris MPI, 445 North Fifth Street, Third Floor, Phoenix, AZ, 85004, US

(21) Appl. No.: 12/211,765

(22) Filed: Sep. 16, 2008

(51) Int. Cl. G01N 33/53; C12Q 1/02; A61K 31/337; A61K 38/38; A61P 35/00

(52) U.S. Cl. 514/12; 435/29; 435/7.8; 514/449

(57) ABSTRACT

Methods for detecting elevated levels of SPARC in biological samples using immunohistochemical staining and therapeutic and diagnostic methods relating to those detection methods.

### FIELD OF THE INVENTION

The present invention generally relates to methods for detecting an overexpression of SPARC (secreted protein, acidic and rich in cysteine) and therapeutic and diagnostic methods relating to those detection methods. More particularly, the present invention is directed to methods for detecting elevated levels of SPARC in biological samples using immunohistochemical staining and therapeutic and diagnostic methods relating to those detection methods.

### BACKGROUND OF THE INVENTION

SPARC is a multifunctional glycoprotein that binds collagen involved in tissue mineralization and repair and extracellular matrix modeling, and is expressed by many different types of cells. It also functions to organize the basement membrane structure that is required for tumor progression. SPARC consists of three modules which each have independent activity and unique properties. Module I is highly acidic, binds calcium ions, interacts with hydroxyapatite, and is involved in the mineralization of cartilage and bone. Module II is cysteine rich and is homologous to a repeated domain in follistatin which binds to activin and inhibin. Module III is the extracellular calcium binding module and had high-affinity calcium binding sites.

In vitro experiments have shown that SPARC disrupts cell adhesion, promotes changes in cell shape, inhibits the cell cycle, regulates cell differentiation, inactivates cellular responses to certain growth factors, and regulates extracellular matrix metalloprotease production. The overexpression of SPARC is present in malignant tumors and correlates with disease progression and poor prognosis. SPARC has also been reported to be localized in the cytoplasm of various cell types.

Immunohistochemistry (IHC) combines several techniques to identify specific tissue components, including the overexpression of various proteins, by utilizing a specific antigen/antibody reaction tagged with a visible label. IHC enables the visualization of the distribution and localization of specific cellular components within a cell or tissue. A typical IHC protocol includes fixing a tissue sample, blocking nonspecific sites in the tissue sample with serum or a blocker protein, incubating the tissue sample with primary antibody, washing the tissue sample buffer, suppressing endogenous peroxidase activity in the tissue sample, washing the tissue sample with buffer, incubating the tissue sample with a HRP-conjugated secondary antibody, washing the tissue sample with buffer, incubating the tissue sample with 3,3′ diaminobenzidine (DAB) substrate, and washing the tissue sample with water. Total time for incubating the tissue sample with the various reagents and carrying out all of the steps is typically over 2 hours.

Due to the correlation between the overexpression of SPARC and malignant tissue, it would be beneficial to have a fast and reliable method for determining whether a sample possesses elevated levels of SPARC. It would also be advantageous to be able to use such a method for diagnostic and therapeutic purposes.

### SUMMARY OF THE INVENTION

The present invention is directed to a fast and reliable method for detecting an elevated level of SPARC in a biological sample which includes immunohistochemical staining. Contrary to typical practice with IHC processes, the method of the present invention does not utilize a protein blocking agent to block nonspecific sites in the sample. In addition, contrary to typical practice, the method of the present invention does not perform the step of inhibiting or suppressing endogenous peroxidase activity in the sample after incubating the sample with a primary antibody and before incubating the sample with peroxidase conjugate. Instead, in the method of the present invention, the step of inhibiting or suppressing endogenous peroxidase activity in the sample is performed prior to incubating the sample with primary antibody. Further, the time frames for exposing various reagents to the sample can be reduced throughout the process thereby resulting in a method for detecting elevated levels of SPARC utilizing IHC which takes between about 1.5 and 1.75 hours to complete.

The method of detecting an elevated level of SPARC in a biological tissue in accordance with the present invention includes: a) providing a slide having a sample contained thereon; b) processing the sample such as deparaffinizing and rehydrating the sample in the event that the sample is a formalin fixed paraffin embedded (FFPE) sample; c) rinsing the sample with buffer and/or other aqueous liquids; d) covering the sample with peroxide, or other suitable oxidative agents for approximately 1 to 60 minutes; e) rinsing the sample with buffer and/or other aqueous liquids; f) covering the sample with a primary SPARC antibody diluted in a Tris based diluent, a negative control reagent, or other suitable carrier solution for approximately 5 to 120 minutes; g) rinsing the sample with buffer and/or other suitable aqueous liquids; h) covering the sample with a secondary detection reagent such as an antibody probe reagent, a secondary binding reagent, or a polymer detection reagent; i) rinsing the sample with buffer and/or other aqueous liquids; j) covering the sample with a horseradish peroxidase conjugate, an enzymatic agent, or a catalytic agent; k) rinsing the sample with buffer and/or other aqueous liquids; l) covering the sample with a first volume of at least one of a 3,3′-diaminobenzidine (DAB) containing solution or a chromogen containing solution for 1 to 60 minutes; m) covering the sample with a second volume of at least one of a DAB containing solution or a chromogen containing solution for 1 to 60 minutes; n) rinsing the sample with water and/or other aqueous liquids; o) covering the sample with a counterstain such as a hematoxyln counterstain for approximately 1 to 20 minutes; p) rinsing the sample with buffer and/or other aqueous liquids; q) repeatedly dipping the sample and the slide in water until the sample is clear; r) dehydrating the sample; s) applying a coverslip over the sample contained on the slide; and t) detecting an elevated level of SPARC by examining the sample under a microscope and comparing the sample to a control.

In one exemplary embodiment, the step of covering the sample with a primary SPARC antibody includes covering the sample with a monoclonal antibody and the step of covering the sample with a secondary detection agent includes covering the sample with a monoclonal antibody probe reagent. Covering the sample with a monoclonal antibody probe reagent may include the step of covering the sample with a Goat Anti-Mouse antibody probe reagent for approximately 20 minutes and covering the sample with a horseradish peroxidase may include the step of covering the sample with a horseradish peroxidase for approximately 20 minutes.

In another exemplary embodiment of the invention, the step of covering the sample with a primary SPARC antibody includes covering the sample with a polyclonal antibody and the step of covering the sample with a secondary detection reagent includes covering the sample with a secondary antibody reagent. Covering the sample with a secondary antibody reagent may include covering the sample with a goat anti-rat immunoglobulin G (IgG) reagent for approximately 15 minutes and covering the sample with a horseradish peroxidase conjugate may include the step of covering the sample with a streptavidin horseradish peroxidase reagent for approximately 15 minutes.

In another aspect of the invention, the step of detecting an elevated level of SPARC may include examining the sample under a microscope and comparing the sample to at least one of a negative control for SPARC and a positive control for SPARC. This step may further include the step of assigning a value to the intensity of the staining for a sample that has an elevated SPARC level. The invention may further include the step of assigning an overall score to a sample with an elevated SPARC level where the overall score is based on the intensity of staining and the percent of cells stained in the sample. Examples of overall scores may include a negative score, a moderately positive score, and a strongly positive score.

In still another exemplary embodiment of the invention, steps c) through p) above may be automated.

The present invention is also directed to a method for treating a malignancy in at least one of a lung, an ovary, a breast, a head, a neck, a pancreas, a colon, a melanocyte, an adrenal cortex, an adipose tissue, and potentially any mammalian tissue which includes the steps of obtaining a sample of the malignancy, detecting whether the sample has an elevated level of SPARC in accordance with steps a) through t) above, and treating the malignancy with at least one of a mitotic inhibitor comprising a taxane or a vinca alkaloid and/or another cytotoxic agents if the sample has an elevated level of SPARC. The mitotic inhibitor may be paclitaxel or paclitaxel bound to albumin.

The method of the present invention for treating a malignancy may also include the step of assigning an overall score to a sample having an elevated level of SPARC where the overall score is based on intensity of staining and the percent of cells stained in the sample, and the step of treating the malignancy with at least one of a mitotic inhibitor and/or another cytotoxic agent is only done when the tissue sample has an overall score that is strongly positive.

The method of the present invention is also directed to a method for diagnosing a malignancy in at least one of a lung, an ovary, a breast, a head, a neck, a pancreas, a colon, a melanocyte, an adrenal cortex, an adipose tissue, and potentially any other mammalian tissue which includes the steps of obtaining a sample from at least one of a lung, an ovary, a breast, a head, a neck, a pancreas, a colon, a melanocyte, an adrenal cortex, an adipose tissue, or any other mammalian tissue, detecting whether the sample has an elevated level of SPARC in accordance with steps a) through t) above, and determining that the sample is malignant if the sample has an elevated level of SPARC. The method for diagnosing a malignancy in at least one of a lung, an ovary, a breast, a head, a neck, a pancreas, a colon, a melanocyte, an adrenal cortex, an adipose tissue, and any other mammalian tissue may also include the step of assigning an overall score to a sample having an elevated level of SPARC where the overall score is based on intensity of staining and the percent of cells stained in the sample, and the step of determining that the sample is malignant is only made if the sample has an overall score that is strongly positive.

### BRIEF DESCRIPTION OF THE DRAWINGS

The present invention will hereinafter be described in conjunction with the appended drawing figures, wherein like numerals denote like elements, and:

FIG. 1 is a flow chart depicting exemplary embodiments of the method of the present invention for detecting an elevated level of SPARC in a biological tissue;

FIG. 2 is a series of photographs showing expression of SPARC in a tissue sample after carrying out the monoclonal and polyclonal methods of the present invention for detecting an elevated level of SPARC compared to a positive control for SPARC and a negative control for SPARC;

FIG. 3 is a table showing results of the exemplary monoclonal and polyclonal embodiments of the present invention for detecting an elevated level of SPARC in a sample set of biological tissues;

FIG. 4 shows a an exemplary scoring chart used for assigning an overall score to a tissue sample after carrying out the methods of the present invention for detecting an elevated level of SPARC;

FIG. 5 is a flow chart depicting an exemplary embodiment of a method for treating a malignancy in accordance with the present invention; and

FIG. 6 is a flow chart showing an exemplary embodiment of a method for diagnosing a malignancy in accordance with the present invention.

### DETAILED DESCRIPTION

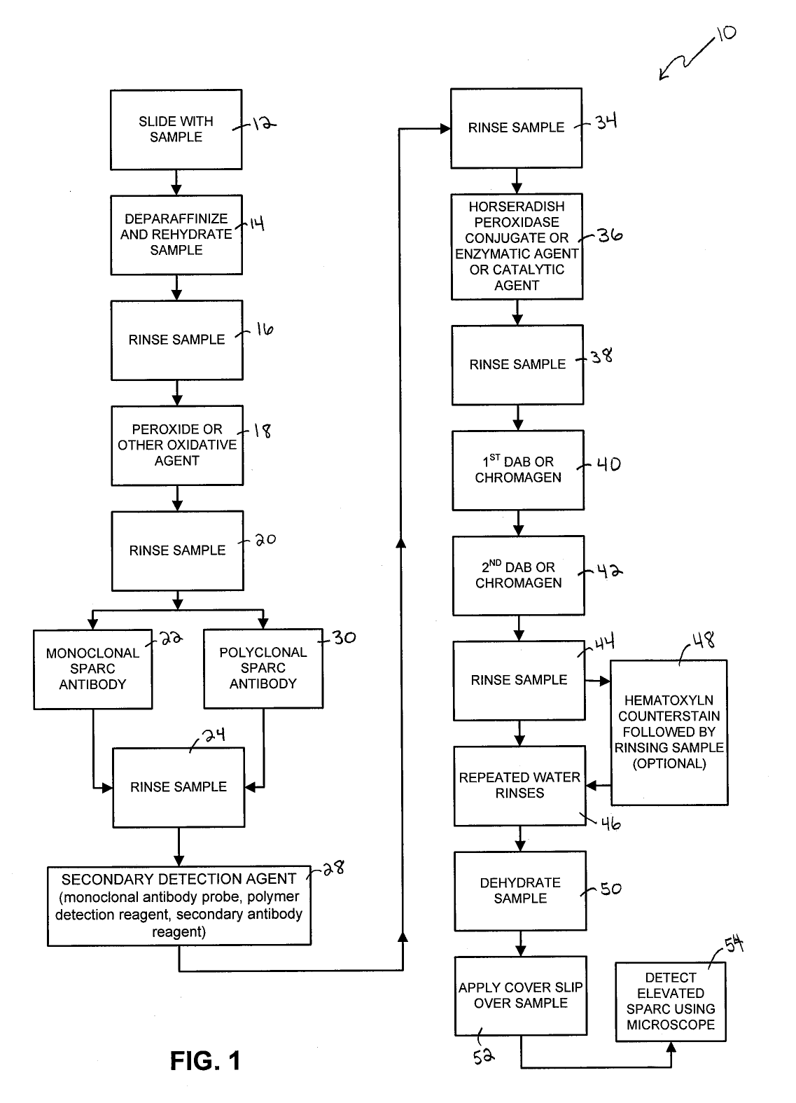

The invention is directed to methods for detecting an elevated level of secreted protein, acidic and rich in cysteine (SPARC) in biological samples using immunohistochemical staining with both monoclonal and polyclonal antibodies. A flow chart depicting exemplary embodiments of the method for detecting overexpression of SPARC is shown in FIG. 1. The method generally includes providing a slide (such as a microscope slide) having a sample contained thereon in step 12 and further processing the sample in step 14 such as deparaffinizing and rehydrating the sample in the event that the sample comprises a formalin fixed paraffin embedded (FFPE) tissue. Other steps for processing the sample may also be performed in step 14 in the event that the sample is a frozen sample or fresh sample. The sample contained on the slide is then rinsed in step 16 with a buffer and/or other aqueous liquids. An exemplary buffer and/or other aqueous liquids used for rinsing the sample may include, but are not limited to, a Tris Buffer.

The sample contained on the slide is then covered with a peroxide solution or other oxidative agent in step 18 to inhibit or block endogenous peroxidase activity in the cells and/or tissues of the sample. In one exemplary embodiment of the invention, the sample is covered in 3% H₂O₂ for approximately five minutes. In other embodiments, other endogenous peroxide blocking agents may be used for a longer period of time including time periods for less than five and time periods up to sixty minutes depending upon the blocking agent.

The sample contained on the slide is then rinsed with buffer and/or other aqueous liquids in step 20 as previously described above with reference to step 16. Following step 20, the sample is covered with a primary SPARC antibody. At this juncture, different SPARC antibodies may be used including monoclonal SPARC antibodies and polyclonal SPARC antibodies. For example, in one exemplary embodiment of the method of the present invention, the sample may be covered with a monoclonal SPARC antibody in step 22 such as, for example, a mouse monoclonal SPARC antibody. However, other SPARC antibodies may be used including, but not limited to those derived from rabbit, goat, human, etc. In the exemplary embodiment utilizing a mouse monoclonal SPARC antibody, the sample is covered with the mouse monoclonal SPARC antibody diluted in a Tris based diluent or a negative control agent for approximately 30 minutes. However, depending upon the type of monoclonal antibody utilized, the sample may be covered with the monoclonal antibody for a period of time ranging between five and 120 minutes. Next, the sample is rinsed in step 24 with a buffer and/or other aqueous liquids as previously described above in step 16. After rinsing, the sample is covered with a secondary detection reagent in step 28. Depending upon the monoclonal SPARC antibody used in step 22, the sample may be covered with a monoclonal antibody probe, a secondary antibody reagent, or a polymer detection reagent for a period of time between 5 and 120 minutes. In the example described above which utilized a mouse monoclonal SPARC antibody in step 22, the sample is covered with a monoclonal antibody probe such as biotinylated goat anti-mouse antibody for twenty minutes in step 28.

In another exemplary embodiment of the method of the present invention, the sample is covered with a polyclonal SPARC antibody in step 30 after being rinsed in step 20. In one exemplary embodiment, the polyclonal SPARC antibody utilized is rat anti-SPARC antibody and is diluted in a Tris diluent or a negative control agent, or other suitable carrier solution, which covers the sample for approximately thirty minutes. Again, depending upon the polyclonal SPARC antibody utilized, a sample may be covered with the polyclonal SPARC antibody for a period of time ranging between five and 120 minutes. Next, the sample is rinsed in step 24 with a buffer or other aqueous liquid as previously described in step 16 and then covered with a secondary detection reagent in step 28. In one exemplary embodiment, the sample may be covered with a polyclonal antibody probe that is a goat anti-rat IgG antibody reagent for approximately 15 minutes. Again, depending upon the type of secondary detection reagent utilized, the sample may be covered with a secondary detection reagent for a period of time ranging between approximately 5 to 120 minutes.

Next, the sample is rinsed in step 34 with a buffer or aqueous liquid as previously described in step 16 after being covered with a secondary detection reagent in step 28. A horseradish peroxidase conjugate, and enzymatic agent, or a catalytic agent is then used to cover the sample in step 36. After rinsing the sample in step 38 with a buffer or other aqueous liquid as previously described above with reference to step 16, the sample is covered with a first volume of a 3,3′-diaminobenzidine (DAB) containing solution and/or a chromagen containing solution in step 40. Next, the sample is covered with a second volume of a DAB containing solution and/or a chromagen containing solution in step 42. Depending upon the solution used, the sample may be covered with a DAB containing solution and/or a chromagen containing solution in steps 40 and 42 for a time period of between approximately one to 60 minutes. In one exemplary embodiment, the sample was covered with two separate volumes of DAB containing solution for approximately ten minutes each with no rinse steps between applying those volumes.

After applying a DAB containing solution and/or a chromagen containing solutions in steps 40 and 42, the sample is rinsed with a buffer or other aqueous liquid in step 44 as previously described above with reference to step 16. After rinse step 44, the sample containing slide is repeatedly rinsed in water in step 46. Step 46 may be performed by repeatedly dipping the sample containing slide into distilled water until the slide appears clear. In addition, an optional step 48 may be carried out which includes covering the sample with a counterstain, such as a hematoxyln counterstain, and then rinsing the sample prior to step 44 in which the sample is repeatedly rinsed with water.

The sample contained on the slide is then dehydrated in step 50 and a cover slip is applied over the sample in step 52 before using a microscope to examine the slide in step 54 so that one examining the slide can detect an elevated level of SPARC.

It will be understood by those of ordinary skill in the art that a number of variations may be made to the steps outlined in FIG. 1 to enable one to detect an overexpression of SPARC. However, in addition to the number of variations which can be made to those steps outlined in FIG. 1, the inventors have conceived of a quick and reliable method for detecting overexpression of SPARC in a biological sample utilizing immunohistochemical staining.

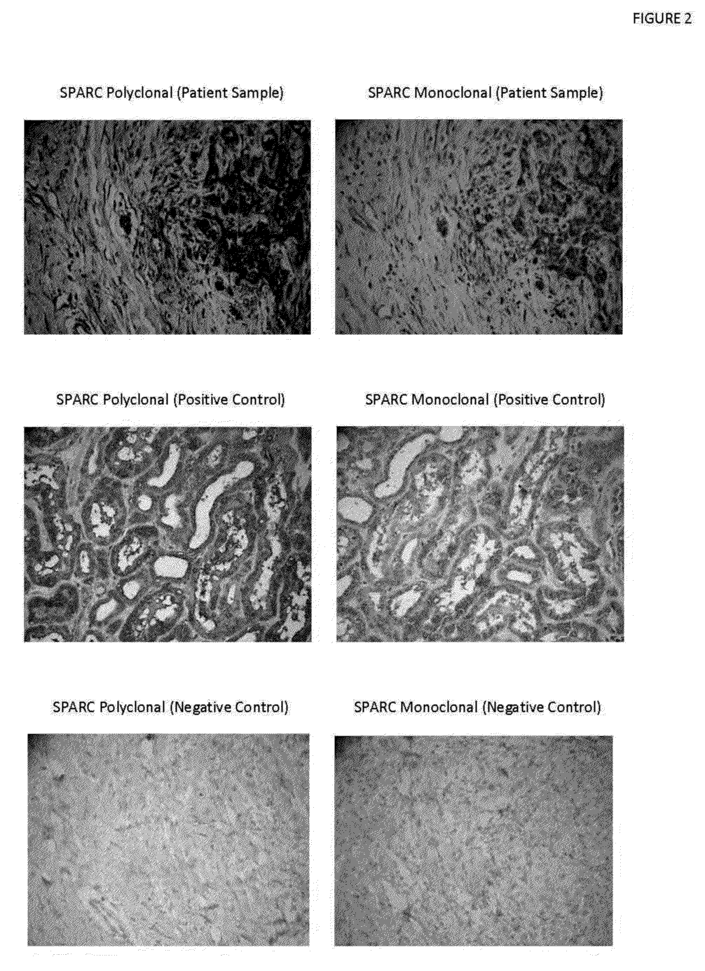

FIG. 2 is a series of photographs showing overexpression of SPARC in patient tissue samples after carrying out the monoclonal and polyclonal methods of the present invention for detecting an elevated level of SPARC compared to a positive control for SPARC and a negative control for SPARC. The method of the present invention for detecting an elevated level of SPARC utilizing a polyclonal antibody is generally described with reference to FIG. 1 but includes the following details regarding some of the method steps: 1) the sample used comprised a formalin fixed paraffin embedded (FFPE) biological tissue; 2) step 14 in FIG. 1 comprised deparaffinizing and rehydrating the FFPE tissue sample; 3) step 18 comprised covering the tissue sample with 3% peroxide for approximately 5 minutes; 4) applying the primary antibody in step 30 comprised covering the tissue sample with a rat Anti-SPARC polyclonal antibody diluted in a Tris based diluent for approximately 30 minutes; 5) applying the secondary antibody reagent in step 32 comprised covering the sample with a goat Anti-Rat IgG antibody reagent for approximately fifteen minutes; 6) step 36 comprised covering the sample with a streptavidin horseradish peroxidase for approximately fifteen minutes; 7) steps 40 and 42 comprised the steps of applying a same volume of DAB containing solution for approximately ten minutes each; and, 8) the sample was covered with a hematoxyln counterstain in step 48 for approximately two minutes.

The method of the present invention for detecting over expression of SPARC utilizing a monoclonal antibody also included the steps described with reference to FIG. 1 along with the following details relating to some of those steps: 1) the sample used comprised a formalin fixed paraffin embedded (FFPE) biological tissue; 2) step 14 in FIG. 1 comprised deparaffinizing and rehydrating the FFPE tissue sample; 3) step 18 comprised covering the tissue sample with 3% peroxide for approximately 5 minutes; 4) applying the primary antibody in step 30 comprised covering the tissue sample with a monoclonal SPARC antibody diluted in a Tris based diluent for approximately 30 minutes; 5) covering the tissue sample in step 28 comprises covering the tissue sample with biotinylated goat anti-mouse antibody for approximately 20 minutes; 6) step 36 comprised covering the sample with a horseradish peroxidase for approximately twenty minutes; 7) steps 40 and 42 comprised the steps of applying a same volume of DAB containing solution for approximately ten minutes each; and, 8) the sample was covered with a hematoxyln counterstain in step 48 for approximately two minutes.

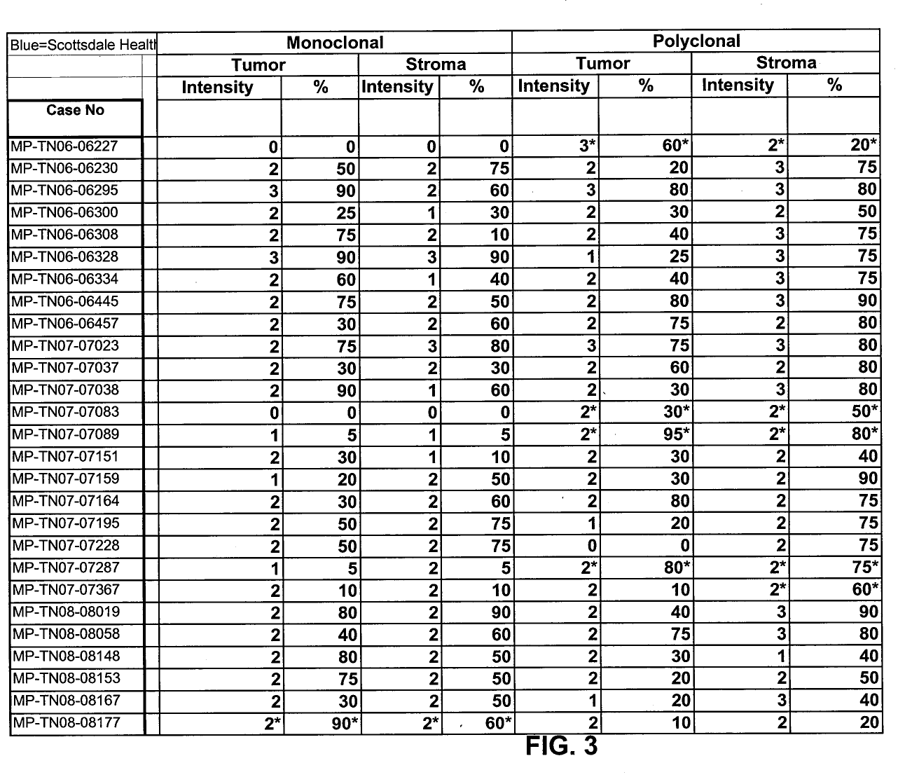

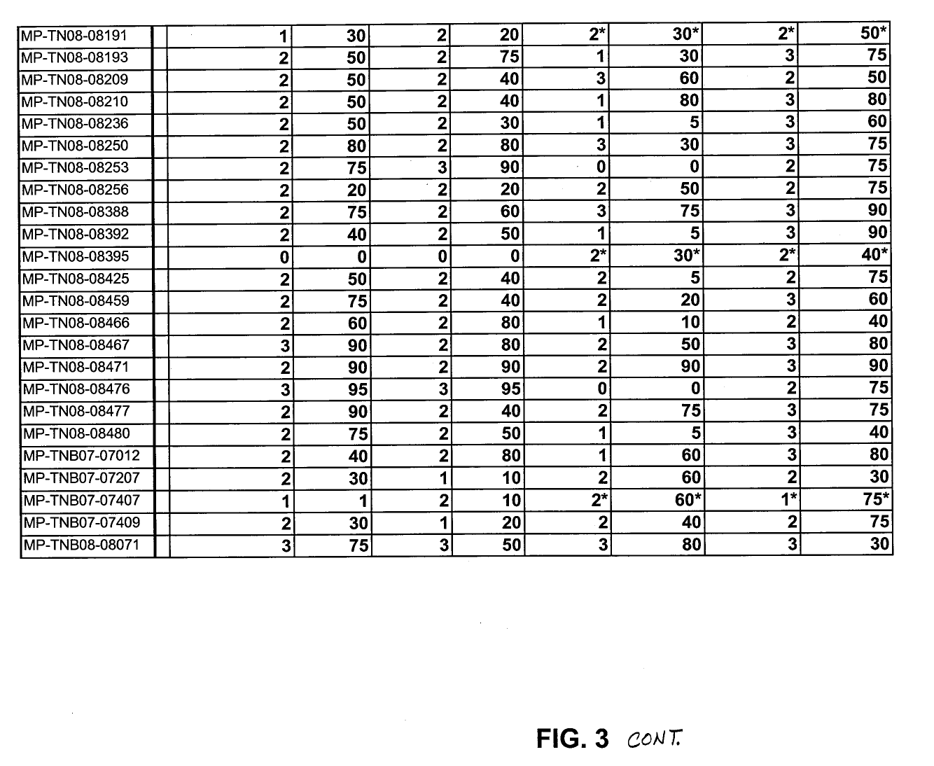

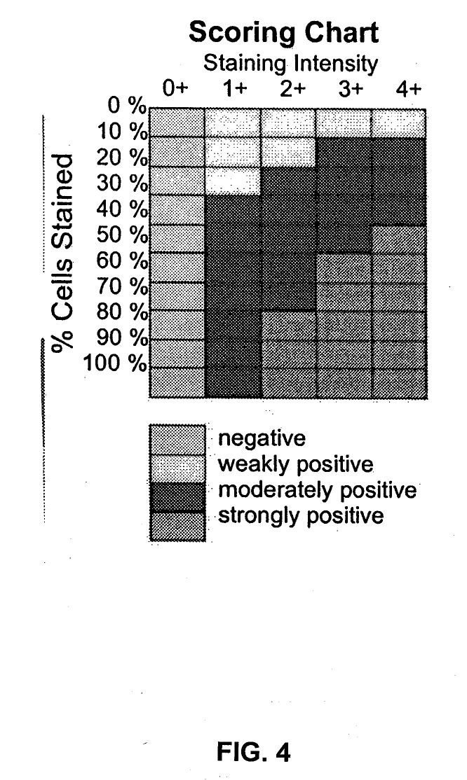

FIG. 3 is a table showing results of the exemplary monoclonal and polyclonal embodiments of the present invention for detecting an elevated level of SPARC in a sample set of biological tissues in accordance with the description of detailed steps outlined in the preceding paragraph. Two different values were applied to both the tumor tissue and the stroma (i.e., the connective tissue surrounding the tumor) for each patient using both the monoclonal method of the present invention for detecting an elevated level of SPARC and the polyclonal method of the present invention for detecting an elevated level of SPARC. An intensity of staining level was applied using a light microscope with the range of staining intensity levels being 0, 1, 2, 3, 4 (also identified as 0+, 1+, 2+, 3+, 4+ in FIG. 4) with 4 being the highest intensity level of staining. A value was also applied for the percent of tumor tissue which was stained and the percent of stroma that was stained with 0% indicating that no tissue was stained and 100% indicating that all of the tissue was stained. A 2+ or more level of staining for 30% or more of the sample tissue indicates overexpression of SPARC.

The asterisks in FIG. 3 identify those tissue samples where the type of antibody singly contributed to the overexpression of SPARC. For example, in the first case identified as MP-TN06-06227, an overexpression of SPARC was noted with the polyclonal method of the present invention for detecting an elevated level of SPARC but no overexpression was identified with the monoclonal method of the present invention for detecting the elevated level of SPARC. Accordingly, the antibody utilized in the polyclonal method of the present invention for detecting an elevated level of SPARC described with reference to FIG. 2 above was singly responsible for identifying the overexpression of SPARC. In another example, the case identified as MP-TN08-08177 in FIG. 3 indicated an overexpression of SPARC when utilizing the monoclonal method of the present invention for detecting an elevated level of SPARC but no overexpression was identified with the polyclonal method of the present invention for detecting an elevated level of SPARC. Accordingly, the antibody used in the monoclonal method of the present invention for detecting an elevated level of SPARC described with reference to FIG. 2 above was singly responsible for identifying the overexpression of SPARC.

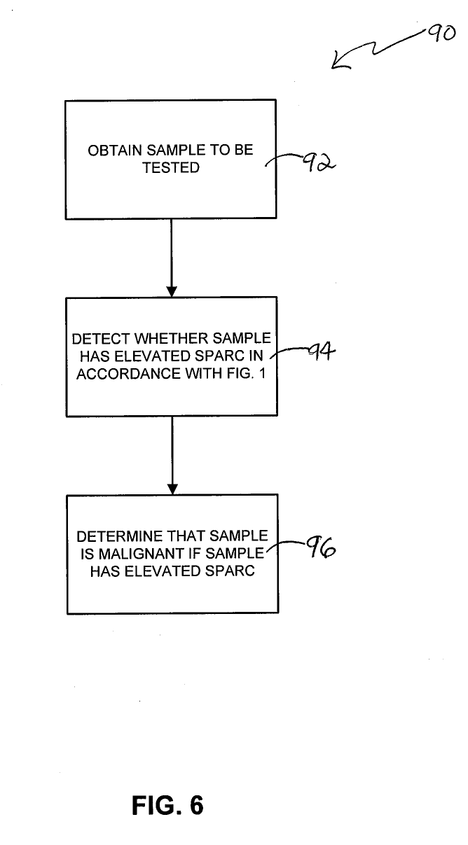

FIG. 6 is an exemplary scoring chart which can be used for assigning an overall score to a sample after carrying out the methods of the present invention for detecting an elevated level of SPARC. The combination of the staining intensity of the sample along with the percent of cells in the sample that was stained correlate to one of four overall scores, namely negative, weakly positive, moderately positive, or strongly positive.

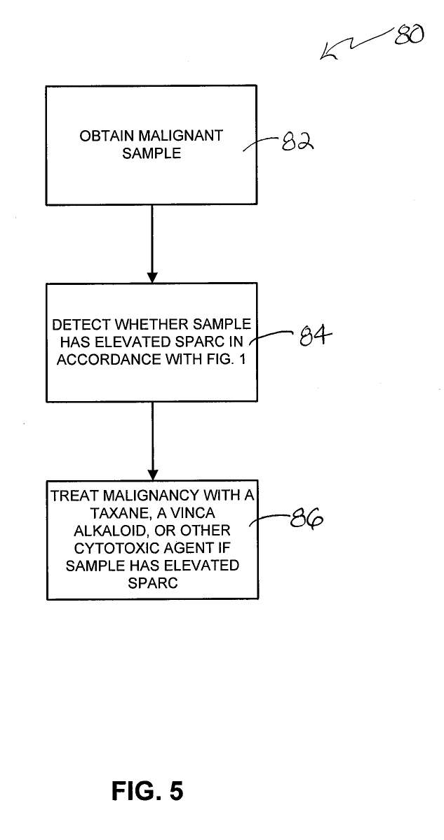

A flow chart depicting an exemplary embodiment of a method for treating a malignancy 80 in accordance with the present invention is shown in FIG. 5. First, a malignant sample is obtained in step 82. Next, a determination is made as to whether the sample possesses an elevated level of SPARC in step 84 by carrying out the method steps shown in, and described with reference to, FIG. 1. Then, if it is determined that the sample has an elevated SPARC level, the patient having the malignancy relating to the sample is treated with a taxane, a vinca alkaloid, and/or some other cytotoxic agent in step 86. Step 86 may include treating a patient's malignancy with paclitaxel or a paclitaxel bound to albumin.

FIG. 6 is a flow chart showing an exemplary embodiment of a method for diagnosing a malignancy 90 in accordance with the methods of the present invention. First, a test sample must be obtained from a patient in step 92. Next, the test sample from the patient is used to determine whether the test sample has an elevated SPARC level by carrying out the method steps shown in, and described with reference to, FIG. 1. Finally, a determination is made that the test sample is malignant if the test sample possesses an elevated level of SPARC in step 96. An elevated level of SPARC is identified as a 2+ or more staining intensity level of 30% or more of the sample.

It will be understood that the foregoing description is of preferred exemplary embodiments of the invention and that the invention is not limited to specific forms shown or described herein. Various modifications may be made in the design, arrangement, order, and types of steps disclosed herein for making and using the invention without departing from the scope of the invention as expressed in the appended claims.

1. A method of detecting an elevated level of SPARC in a biological tissue comprising the steps of:
a) providing a slide having a fixed sample contained thereon;
b) deparaffinizing and rehydrating or further processing the sample for histochemistry;
c) rinsing the sample with at least one of a buffer and another aqueous liquid;
d) covering the sample with a peroxide or other oxidative agent for approximately 1 to 60 minutes;
e) rinsing the tissue with at least one of a buffer and another aqueous liquid;
f) covering the sample with at least one primary SPARC antibody diluted in a Tris based diluent, a negative control reagent, or another suitable carrier solution for approximately 5 to 120 minutes without prior or subsequent application of a blocking agent to the sample;
g) rinsing the sample with at least one of a buffer and another aqueous liquid;
h) covering the sample with at least one secondary detection reagent;
i) rinsing the sample with at least one of a buffer and another aqueous liquid;
j) covering the sample with a horseradish peroxidase conjugate, an enzymatic agent, or other catalytic agent;
k) rinsing the sample with at least one of a buffer and another aqueous liquid;
l) covering the sample with a first volume of at least one of a 3,3′-diaminobenzidine containing solution and a chromagen containing solution for approximately 1 to 60 minutes;
m) covering the sample with a second volume of at least one of a 3,3′-diaminobenzidine containing solution and a chromagen containing solution for approximately 1 to 60 minutes;
n) rinsing the sample with at least one of water and another aqueous liquid;
o) covering the sample with a hematoxyln counterstain for approximately 2 minutes;
p) rinsing the sample with at least one of a buffer and another aqueous liquid;
q) repeatedly dipping the sample and the slide in distilled water until the slide is clear;
r) dehydrating the sample;
s) applying a cover slip over the sample contained on the slide; and
t) detecting an elevated level of SPARC by examining the sample under a microscope and comparing the sample to a control.

2. The method of claim 1 wherein the step of covering the sample with at least one primary SPARC antibody comprises the step of covering the sample with one or more monoclonal antibodies and the step of covering the sample with at least one secondary detection reagent comprises the step of covering the sample with one or more monoclonal antibody probe reagents.

3. The method of claim 2 wherein the step of covering the tissue with one or more monoclonal antibody probe reagents comprises the step of covering the tissue with a mouse monoclonal antibody probe reagent for approximately 20 minutes and the step of covering the sample with a horseradish peroxidase conjugate, and enzymatic agent, or other catalytic agent comprises the step of covering the tissue with a horseradish peroxidase for approximately 20 minutes.

4. The method of claim 1 wherein the step of covering the tissue with at least one primary SPARC antibody comprises the step of covering the sample with one or more polyclonal antibodies and the step of covering the sample with at least one secondary detection reagent comprises the step of covering the sample with one or more polyclonal antibody probe reagents.

5. The method of claim 4 wherein the step of covering the sample with one or more polyclonal antibody probe reagents comprises the step of covering the sample with a goat anti-rat immunoglobulin G reagent for approximately 15 minutes and the step of covering the sample with a horseradish peroxidase conjugate, and enzymatic agent, or other catalytic agent comprises the step of covering the sample with a streptavidin horseradish peroxidase reagent for approximately 15 minutes.

6. The method of claim 1 wherein the step of detecting an elevated level of SPARC comprises the step of examining the sample under a microscope and comparing the sample to at least one of a negative control for SPARC and a positive control for SPARC.

7. The method of claim 6 further comprising the step of assigning a value to the intensity of staining for a sample that has an elevated level of SPARC.

8. The method of claim 7 further comprising the step of assigning an overall score to a sample that has an elevated level of SPARC wherein the overall score is based on the intensity of staining and the percent of cells stained in the sample.

9. The method of claim 8 wherein the overall score comprises at least one of a negative score, a moderately positive score, and a strongly positive score.

10. The method of claim 1 wherein at least steps c) through p) are automated such that they do not require manual handling of the samples and slides.

11. A method for treating a malignancy in at least one of a lung, an ovary, a breast, a head, a neck, a pancreas, a colon, a melanocyte, an adrenal cortex, an adipose tissue, and any other mammalian tissue which comprises:
obtaining a sample of the malignancy;
detecting whether the sample has an elevated level of SPARC in accordance with claim 1; and
treating the malignancy with at least one of a mitotic inhibitor comprising a taxane or a vinca alkaloid and another cytotoxic agent if the sample has an elevated level of SPARC.

12. The method of claim 11 wherein the mitotic inhibitor comprises paclitaxel.

13. The method of claim 12 wherein the mitotic inhibitor comprises paclitaxel bound to albumin.

14. A method for treating a malignancy in at least one of a lung, an ovary, a breast, a head, a neck, a pancreas, a colon, a melanocyte, an adrenal cortex, an adipose tissue, and any other mammalian tissue which comprises:
obtaining a sample of the malignancy;
detecting whether the sample has an elevated level of SPARC in accordance with claim 9; and
treating the malignancy with a mitotic inhibitor comprising at least one of a taxane or a vinca alkaloid and another cytotoxic agent if the sample has an overall score that is strongly positive.

15. The method of claim 14 wherein the mitotic inhibitor comprises paclitaxel.

16. The method of claim 15 wherein the mitotic inhibitor comprises paclitaxel bound to albumin.

17. A method for diagnosing a malignancy in at least one of a lung, an ovary, a breast, a head, a neck, a pancreas, a colon, a melanocyte, an adrenal cortex, an adipose tissue, and any other mammalian tissue which comprises:
obtaining a sample from at least one of a lung, an ovary, a breast, a head, a neck, a pancreas, a colon, a melanocyte, an adrenal cortex, an adipose tissue, and any other mammalian tissue;
detecting whether the sample has an elevated level of SPARC in accordance with claim 1; and
determining that the sample is malignant if the sample has an elevated level of SPARC.

18. A method for diagnosing a malignancy in at least one of a lung, an ovary, a breast, a head, a neck, a pancreas, a colon, a melanocyte, an adrenal cortex, an adipose tissue, and any other mammalian tissue which comprises:
obtaining a sample from at least one of a lung, an ovary, a breast, a head, a neck, a pancreas, a colon, a melanocyte, an adrenal cortex, an adipose tissue, and any other mammalian tissue;
detecting whether the sample has an elevated level of SPARC in accordance with claim 9; and
determining that the sample is malignant if the tissue sample has an overall score that is strongly positive.
